(19) United States
(12) Patent Application Publication
(10) Pub. No.: US 2011/0033392 A1
(43) Pub. Date: Feb. 10, 2011

(54) DEODORIZING COMPOSITION UNDER WEAK ACIDITY

(75) Inventors: Atsushi Narise, Kawasaki-shi (JP); Takahito Takase, Asaka-shi (JP); Sanae Kikuchi, Saitama-shi (JP); Kenji Osawa, Saitama-shi (JP)

Correspondence Address: MILES & STOCKBRIDGE PC, 1751 PINNACLE DRIVE, SUITE 500, MCLEAN, VA, 22102-3833, US

(73) Assignee: LOTTE CO., LTD

(21) Appl. No.: 12/866,505

(22) PCT Filed: Feb. 6, 2009
(86) PCT No.: PCT/JP2009/052445

§ 371 Date: Aug. 6, 2010

(30) Foreign Application Priority Data
Feb. 13, 2008 (JP) 2008-0313182

(51) Int. Cl. A61K 9/68; A61K 38/44; A61Q 15/00

(52) U.S. Cl. 424/48; 424/94.4

(57) ABSTRACT

The present invention provides a deodorizing composition containing as an active ingredient thereof a natural plant extract with high safety, which can be used for foods without any anxiety concerning safety in use and shows a high deodorizing effect even in a weakly acidic condition, and foods and drinks containing such a composition. The deodorizing composition comprises a plant of the genus Rubus of the family Rosaceae, laccase and an acid.

### TECHNICAL FIELD

The present invention relates to a deodorizing composition containing laccase and an extract of a plant of the genus Rubus of the family Rosaceae under a weakly acidic condition, and the foods and drinks containing such a deodorizing composition.

### BACKGROUND ART

As means for removing or killing an offensive odor, there have conventionally been used such methods as masking with an aromatic material, chemical deodorization by use of an oxidizer, counteragent, fixative or the like, or adsorption by active carbon. Any of these methods, however, is in many cases subject to strict restrictions depending on the purpose of use. Particularly in the field of foodstuff, although the deodorizers containing cyclodextrin, chlorophylls and some types of plant extracts as an active ingredient have been patented, there have remained the unsolved problems such as too large adverse influence on the flavor and feel to the palate of the food to which a deodorizer has been added, due to its specific color, smell and taste such as bitterness or astringency, and insufficient deodorizing effect. As the deodorizers having an excellent deodorizing effect to solve the above problems, the attention is focused on the crude drugs and natural plant extracts such as herb which are free of side effects and high in safety and have been popularly used since old times. From among these products, the extracts of the plants of the genus Rubus of the family Rosaceae which exhibit a strong deodorizing effect have been discovered, and their practical applications are being studied.

It is known that the plants of the genus Rubus of the family Rosaceae show a strong deodorizing effect against methyl mercaptan which is a thiol compound, trimethylamine which is a nitrogen compound, and allylmethyl monosulfide which is a monosulfide compound. The deodorizing compositions containing as an active ingredient Tien-cha which is a plant of the same genus as Rubus are also known as a deodorizing agent. Extracts of the plants of the genus Rubus of the family Rosaceae exhibit a high deodorizing effect and are applied to various kinds of foods and drinks including confectionary because of their high safety characteristics.

Extracts of the plants of the genus Rubus of the family Rosaceae, however, have a drawback that their deodorizing effect is excessively lowered in a weakly acidic condition. In order to enhance the deodorizing effect of extracts of the plants of the genus Rubus of the family Rosaceae in a food or drink under a weakly acidic condition, it is necessary to use the extract in an amount far over the ordinary proper level, but this would lead to a high price of the deodorizing composition and is unfavorable from the viewpoint of practical applicability.

As a result of concentrated studies on the subject matter, the present inventors found that the combined use of laccase, which is an enzyme, and an extract of the plants of the genus Rubus of the family Rosaceae (extract of Tien-cha, blackberry or raspberry) provides a remarkable improvement of the deodorizing activity under a weakly acidic condition (pH 4.0 to 7.0).

Many studies have been conventionally made on the deodorizing effect by the combined use of a plant extract and an enzyme.

For instance, the theses on the deodorizing effect by the combined use of plant extracts and polyphenol oxidase are presented in Food Science and Technology Research, 5(2), 176-180, 1999; Journal for the Integrated Study of Dietary Habits, 10(3), 15-19, 1999; The Takasago times, No. 133, p6-14 (1999, 12. 05); and Biosci. Biotech. Biochem, 61(12), 2080-2084, 1997. In Food Science and Technology Research, 5(2), 176-180, 1999; Journal for the Integrated Study of Dietary Habits, 10(3), 15-19, 1999; The Takasago times, No. 133, p6-14 (1999, 12. 05); and Biosci. Biotech. Biochem, 61(12), 2080-2084, 1997, there are disclosed the deodorizing effect and the deodorizing mechanism by the combined use of vegetables, fruits, mushrooms and an enzyme (polyphenol oxidase). Any of these theses, however, is silent on the deodorizing effect by the combined use of an extract of the plants of the genus Rubus of the family Rosaceae and laccase.

Further, the theses on the deodorizing effect by the combined use of plant extracts and polyphenol oxidase are presented in the above Food Science and Technology Research, 5(2), 176-180, 1999; Journal for the Integrated Study of Dietary Habits, 10(3), 15-19, 1999; The Takasago times, No. 133, p6-14 (1999. 12. 05); Biosci. Biotech. Biochem, 61(12), 2080-2084, 1997; and Journal of Esthetic Dentistry, Vol. 17, No. 1, p90-(2004. 09). Presented in Journal of Esthetic Dentistry, Vol. 17, No. 1, p90-94 (2004. 09), is a thesis relating to improvement in the deodorizing effect by the combination of a rosemary extract and laccase, and it states that the above combination provided enhancement of deodorizing performance in a pH range of 4.5 to 6.0. This thesis, however, makes no mention of the deodorizing effect by the combination of an extract of the plants of the genus Rubus of the family Rosaceae and laccase.

Researches on the deodorizing effect by the combined use of a mushroom extract, ku-ding-cha and polyphenol oxidase are reported in J. Agric. Food Chem. 2001, 49(11), 5509-5514; and J. Agric. Food Chem. 2004, 52(17), 5513-5518. J. Agric. Food Chem. 2001, 49(11), 5509-5514 is a thesis on the methyl mercaptan capturing ability of the mushroom extracts containing polyphenol. J. Agric. Food Chem. 2004, 52(17), 5513-5518 is a thesis on the deodorizing effect by the combined use of an ku-ding-cha, green tea, black tea or oolong tea extracts and polyphenol oxidase derived from fruit. In any of these literatures, however, no mention is made of the deodorizing effect by the combination of an extract of the plants of the genus Rubus of the family Rosaceae and laccase.

Literatures relating to the deodorizing effect of the plants of the genus Rubus of the family Rosaceae are given in Japanese Patent Publication No. H05-36061, Japanese Patent Application Laid-Open No. 2003-335647, and Japanese Patent No. 3,633,634. Japanese Patent Publication No. H05-36061 and Japanese Patent Application Laid-Open No. 2003-335647 disclose deodorizing activity against trimethylamine, methyl mercaptan and allylmethyl monosulfide by the extracts of Tien-cha, raspberry and blackberry which are the plants of the genus Rubus of the family Rosaceae. Japanese Patent No. 3,633,634 discloses the deodorizing agent from the Tien-cha extract against methyl mercaptan and trimethylamine. Any of these patents, however, make no mention of the remarkable improvement of the deodorizing effect provided by the combination of an extract of the plants of the genus Rubus of the family Rosaceae and laccase in a weakly acidic condition.

Literatures relating to the deodorizing efficacy of such enzymes as polyphenol oxidase and laccase are presented by Japanese Patent Application Laid-Open No. 2004-148046, Japanese Patent Application Laid-Open No. H09-038193 (Japanese Patent No. 3,562,668), Japanese Patent Application Laid-Open No. 2003-175095 (Japanese Patent No. 3,766,375), Japanese Patent Application Laid-Open No. H10-212221 (Japanese Patent No. 3,625,976), Japanese Patent Application Laid-Open No. 2001-095910 (Japanese Patent No. 3,741,914) and Japanese Patent Application Laid-Open No. 2003-009784. Japanese Patent Application Laid-Open No. 2004-148046 discloses a deodorant composition comprising a combination of soluble lignin and polyphenol oxidase, and Japanese Patent Application Laid-Open No. H09-038183 (Japanese Patent No. 3,562,668) discloses a deodorizer composition comprising a combination of a phenolic compound and polyphenol oxidase. Japanese Patent Application Laid-Open No. 2003-175095 (Japanese Patent No. 3,766,375) and Japanese Patent Application Laid-Open No. H10-212221 (Japanese Patent No. 3,625,976) discloses a deodorant composition comprising a combination of an extract of tea, rosemary, sunflower seeds or raw coffer beans and polyphenol oxidase, and Japanese Patent Application Laid-Open No. 2001-095910 (Japanese Patent No. 3,741,914) discloses a deodorant composition comprising a combination of a glycoside of a phenolic compound and laccase. Japanese Patent Application Laid-Open No. 2003-009784 discloses foods and drinks having a deodorizing effect characterized by containing pulp obtained from a milled plant liquid having a redox enzyme activity. Any of these literatures, however, makes no reference to the fact that the deodorizing effect is remarkably improved under a weakly acidic condition by use of an extract of the plants of the genus Rubus of the family Rosaceae in combination with laccase.

A literature relating to application of laccase to confectionary such as gum, candies, tablets and gummi candies is given in Japanese Patent Application Laid-Open No. 2004-321077. This patent discloses chewing gum characterized by containing laccase-filled capsules and a rosemary extract. In this patent, however, no reference is made to the remarkable improvement of deodorizing effect in a weakly acidic condition by use of an extract of the plants of the genus Rubus of the family Rosaceae in combination with laccase.

As viewed above, although there exist literatures reporting improvements of deodorizing effect by the combined use of plant extracts and enzymes, there is yet no literature disclosing or suggesting the possibility of providing remarkable improvement of the deodorizing effect, even in a weakly acidic condition, by the use of extracts of the plants of the genus Rubus of the family Rosaceae in combination with laccase which is an enzyme.

### DISCLOSURE OF THE INVENTION

The present invention aims at solving the technical problem that the deodorizing activity of extracts of the plants of the genus Rubus of the family Rosaceae lowers remarkably under a weakly acidic condition. Also, the present invention is capable of providing the foods and drinks having both sourness and a deodorizing function. Further, according to the present invention, it is made possible to provide the weakly acidic oral care products having a deodorizing ability, the weakly acidic skin-care or hair-care products having a deodorizing ability, and the weakly acidic deodorants.

The conventional acid-incorporated products such as fruit gum, candies and tablets are reduced remarkably in activity of the deodorizing material (Tien-cha) when placed in a weakly acidic condition. As a result of researches for a material capable of producing a deodorizing effect under a weakly acidic condition, it was confirmed that the deodorizing activity can be enhanced remarkably under a weakly acidic condition (pH 4 to 7) by using an extract of the plants of the genus Rubus of the family Rosaceae (Tien-cha, blackberry or raspberry) in combination with laccase.

According to the present invention, there is provided a deodorizing composition comprising a plant of the genus Rubus of the family Rosaceae, laccase and an acid.

There is also provided the above-described deodorizing composition in which the plant of the genus Rubus of the family Rosaceae is one or more extracts selected from the group consisting of the extracts of Tien-cha, blackberry and raspberry.

The present invention also provides the above-described deodorizing composition in which the acid is selected from the group consisting of citric acid, lactic acid, malic acid, tartaric acid, fumaric acid and acetic acid.

According to the present invention, there is further provided the above-described deodorizing composition in which pH in the saliva after intake of the deodorizing composition is in the range of 4.0 to 7.0 inclusive.

Also provided according to the present invention are chewing gum, candies, tablets and gummi candies comprising a deodorizing composition mentioned the above.

### BRIEF DESCRIPTION OF THE DRAWINGS

FIG. 1 shows the relation between methyl mercaptan deodorization rate and relative activity of enzyme.

FIG. 2 shows the result of a test on the deodorizing effect of a tablet embodying the present invention.

### BEST MODE FOR CARRYING OUT THE INVENTION

The present invention is described in detail below.

The deodorizing composition of the present invention has a plant of the genus Rubus of the family Rosaceae as a main ingredient.

Examples of the plants of the genus Rubus of the family Rosaceae usable in the present invention include raspberry (Rubus idaeus), blackberry (Rubus fruticosus), Kaji-ichigo (Rubus trifidus), black raspberry (Rubus occidentalis), and Tien-cha (Rubus suavissimus). The pericarp, leaf, sarcocarp, fruit, wood, bark and root, preferably leaf, of the plants of the genus Rubus of the family Rosaceae are used after dried. The method of obtaining the extracts of the above plants, which are used as a main ingredient of the composition of the present invention, is not specifically limited; for instance, it is possible to use a method in which a plant such as mentioned above is ground by an appropriate grinding means and then subjected to solvent extraction including two-stage extraction. As the extraction solvent, water, lower alcohols such as methanol, ethanol, n-propanol and n-butanol, and other organic solvents such as ethers, chloroform, ethyl acetate, acetone, glycerin and propylene glycol can be used either singly or in the form of a mixture of two or more thereof. Preferably, water or a hydrophilic organic solvent is used. In view of probable usage of the extracts of this invention in foods and drinks, a combination of water and ethanol is preferably used as the extraction solvent from the aspect of safety.

As for the extraction conditions, it is possible to carry out extraction over a wide range of temperature from a high to a low temperature including room temperature, but preferably extraction is conducted at a temperature of from 50 to 90° C. for a period of approximately one to 5 hours. The obtained extract is filtered, and after evaporating away the extracted solvent, it may be concentrated or lyophilized under reduced pressure. Also, the extract may be subjected to fractional purification with an organic solvent or by column chromatography.

The deodorizing composition of the present invention has good fragrance and taste and is also credited with high safety, so that it can be used, for instance, as a deodorizing composition for such products as gargles, tooth pastes, deodorant sprays and the like, or may be blended in confectionery such as chewing gum, candies, tablets, gummi jellies, chocolates, biscuits and snacks; frozen dessert such as ice cream, sherbet and ice candies; beverages, bread, hotcakes, dairy products, livestock products such as ham and sausage; fish meat products such as kamaboko (boiled fish paste) and chikuwa (a kind of fish paste); and other foods and drinks such as household dish, custard pudding, soup and jam. Thus, the composition of the present invention can find everyday dietary use. The use of the composition of the present invention is not limited to the edible products; the deodorizing composition can be also blended in such products as soap, shampoo, rinse, skin cream, skin lotion, deodorants for pets, interior deodorants, interior cleaning filters, deodorants for toilet rooms, etc., whereby it is possible to provide the acidic products with an excellent deodorizing activity.

The contents of the ingredients are variable depending on the various producing conditions. The deodorizing composition contains a plant of the genus Rubus of the family Rosaceae in an amount of 0.01% by weight or more and 1.0% by weight or less, preferably 0.05% by weight or more and 0.4% by weight or less. Laccase, whose content is also variable depending on the titer of the laccase preparation used, is added in an amount of 0.01% by weight or more and 1.0% by weight or less, preferably 0.015% by weight or more and 0.12% by weight or less.

When the amount of laccase added is represented by titer, laccase is blended in an amount of 5.90×10² U or more and 5.9×10⁴ U or less, preferably 3.0×10³ U or more and 2.4×10⁴ U or less, per 100 g of the deodorizing composition.

Here, with reference to titer of laccase, the following matter is to be noted. The enzyme activity required for increasing, by 0.001 in a period of 30 seconds, the absorbance at 420 nm of the condensate obtained from an oxidative condensation reaction of chlorogenic acid catalyzed by laccase at 37° C. is supposed to be 1 U. 100 μl of a 50 mM chlorogenic acid solution (0.1 M sodium phosphate buffer solution (pH 6.0)) was added to 500 μl of an enzyme solution (0.1 M sodium phosphate buffer solution (pH 6.0)) and reacted at 37° C. for 5 minutes. Then 500 μl of 0.1 N sulfuric acid was added to stop the reaction, and absorbance at 420 nm was measured. The enzyme activity needed for increasing, by 0.001, the absorbance at 420 nm 30 seconds after the start of the reaction was defined as 1 U (unit).

### EXAMPLES

The present invention is described in further detail below by showing the embodiments thereof, which embodiments, however, are not to be construed as limiting the scope of the invention.

### Example 1

### Preparation of Samples

### Example 1-1

### Laccase Preparation

As a laccase preparation, “Laccase Daiwa Y120” (trade name, produced by Daiwa Chemical Industries Ltd.; titer: 5.9×10⁴ U/g or above) was used in its as-is powdery form or after dissolved in a phosphate buffer solution. The enzyme ratio in Laccase Daiwa Y120 was 30%, the remainder being dextrin.

### Example 1-2

Plants Used

In the Examples shown below, Tien-cha (Rubus suavissimus), blackberry (Rubus fruticosus) and raspberry (Rubus idaeus) were used as the plants of the genus Rubus of the family Rosaceae, and green tea was used as control.

### Example 1-3

### Preparation of Tien-Cha Extract

A two-stage extraction product obtained by the following method was used as a Tien-cha extract.

To 30 g of dry powder of Tien-cha, 300 ml of 100% ethanol was added as a pretreating agent, and with a reflux condenser attached, the mixture was extracted under reflux at 60° C. for one hour. 300 ml of water was added successively to the extraction residue obtained by filtration, and with a reflux condenser attached, it was further extracted under reflux at 90° C. for one hour.

The resulting extract solution was filtered and, after removing the solvent, freeze-dried to obtain 6.4 g of Tien-cha extract (yield: 21%).

### Example 1-4

### Preparation of Blackberry, Raspberry and Green Tea Extracts

300 ml of water was added to 30 g of dry powder of blackberry leaf, and with a reflux condenser attached, the solution was extracted under reflux at 90° C. for one hour. The resultant extract solution was filtered and, after removing the solvent, freeze-dried to obtain 5.2 g of blackberry extract (yield: 17.3%).

In the same way as described above, extraction was carried out using 30 g of dry powder of raspberry leaf and 30 g of dry powder of green tea to obtain 5.2 g of raspberry extract (yield: 17.3%) and 9.8 g of green tea extract (yield: 32.7%).

### Example 1-5

### Preparation of Tablets Containing Tien-Cha Extract, Citric Acid and Laccase

The materials were mixed according to the formulation shown in Table 1 below, and the tablets were obtained therefrom by a conventional method.

TABLE 1

| | Example tablets (wt %) |
|---|---|
| Dextrose | 92 |
| Sugar ester | 4 |
| Flavor | 1 |
| Tien-cha extract | 0.01-0.4 |
| Citric acid | 0-2 |
| Laccase preparation | 0.01-0.4 |
| (5.9 × 10⁴ U/g) | |
| Water | Balance |

### Example 1-6

### Preparation of Tablets Containing Blackberry Extract, Citric Acid and Laccase

The materials were mixed according to the formulation shown in Table 2 below, and the tablets were obtained therefrom by a conventional method.

TABLE 2

| | Example tablets (wt %) |
|---|---|
| Dextrose | 92 |
| Sugar ester | 4 |
| Flavor | 1 |
| Blackberry extract | 0.2 |
| Citric acid | 2 |
| Laccase preparation | 0.1 |
| (5.9 × 10⁴ U/g) | |
| Water | Balance |

### Example 1-7

### Preparation of Chewing Gum Containing Tien-Cha Extract, Citric Acid and Laccase

The materials including a prepared Tien-cha extract, laccase preparation and citric acid were mixed according to the formulation shown in Table 3 and processed according to a conventional method to obtain Example chewing gum.

Control chewing gum not containing laccase preparation and citric acid but containing a prepared Tien-cha extract alone as a deodorizing component was also obtained according to a conventional method.

TABLE 3

| | Control chewing | Example chewing |
|---|---|---|
| | gum (wt %) | gum (wt %) |
| Gum base | 20 | 20 |
| Xylitol | 45 | 45 |
| Maltitol | 33 | 33 |
| Flavor | 2 | 2 |
| Total | 100 | 100 |

The following materials were added
to the above formulation

| | | |
|---|---|---|
| Tien-cha extract | 0.25 | 0.25 |
| Citric acid | — | 1.0 |
| Laccase | — | 0.2 |
| preparation | | |
| (5.9 × 10⁴ U/g) | | |

### Example 2

### Deodorization Testing Method

### Example 2-1

### Enzymatic Deodorization Testing Method with Laccase

1 to 2.5 mg of an extract of the plants of the genus Rubus of the family Rosaceae was weighed out precisely as a sample and put it into a vial, to which 1 ml of a phosphate buffer solution (pH 4.0 to 7.0) was added, allowing the extract to be well dissolved or dispersed therein. Then 100 μl of a laccase solution was added, followed by further addition of 500 μl of a 25 ppm sodium methyl mercaptide solution, after which the vial was closed by a Teflon (registered trademark)—coated rubber stopper and the mixture was reacted at 37° C. for 5 minutes. 150 μl of the headspace gas in the vial after the reaction was injected into a gas chromatograph equipped with an FPD detector, and the amount of methyl mercaptan was calculated from the obtained peak height.

### (Evaluation of Deodorizing Activity)

The amount of methyl mercaptan (M_{S}) when a sample extract of the plants of the genus Rubus of the family Rosaceae was added and the amount of methyl mercaptan (M_{B}) when the extract was not added were measured, and the methyl mercaptan deodorization rate was calculated from the following equation.

Methyl mercaptan deodorization rate (%)=(M_{B}−M_{S})/M_{B}×100

### Example 2-2

### Deodorization Test 1 on Prepared Tablet

2 g of prepared tablet was weighed out precisely as a sample and put it into a vial, to which 8 ml of a 0.2 M phosphate buffer solution (pH 7.5) was added, allowing the tablet to be well dissolved therein to prepare a testing solution. 500 μL of a 25 ppm sodium methyl mercaptide solution was added to 1 ml of the testing solution, and after closing the vial with a Teflon (registered trademark)-coated rubber stopper, the mixture was reacted at 37° C. for 5 minutes. 150 μl of the head space gas in the vial after the reaction was injected into a gas chromatograph equipped with an FPD detector, and the amount of methyl mercaptan was calculated from the obtained peak height.

### Example 2-3

### Deodorization Test 2 on Prepared Tablet

The subjects for the test were prohibited from taking any food and drink except for the sample, conducting tooth cleaning and smoking during the period from rising in the morning till the end of the test. Collection of the breath was started sometime between 9 and 9:30 A.M. Each subject was asked to breathe through his or her nose in repose for 30 seconds with a breath collecting syringe being held in his or her mouth. 0.5 ml of breath was collected, and the amounts of VSC (hydrogen sulfide, methyl mercaptan and dimethyl monosulfide) in the breath were assayed by an Oral Chroma™ (mfd. by Abimedical Co., Ltd.). Halitosis of each subject was measured at the time of rising, then each subject was allowed to wash his or her mouth (15 ml, one minute) or to take a prepared tablet (one tablet), and then the amounts of VSC in the breath were again measured. Thereafter, the breath was collected and examined at intervals of 30 minutes. Collection of breath was continued till the passage of 120 minutes after intake of the sample, and the amounts of VSC in the breath were measured.

### Example 3

### Organoleptic Evaluation Method

Organoleptic evaluation was conducted by collecting the Halitosis from 5 subjects 10 minutes before start of the test, letting each subject take 6 pieces of fried dumpling over a period of 2 minutes, then letting him or her take (masticate for 5 minutes) a prepared Example chewing gum and a control one, or him or her to gargle (twice with 20 ml of water), collecting the breath of each subject in a bag immediately after intake of the gum, and determining the degree of halitosis. Organoleptic evaluation was made according to the standards for judgment shown in Table 4 below. The measurements by 5 panelists were averaged and expressed numerically.

TABLE 4

| Score | Standards for judgment |
|---|---|
| 5 | Very strong odor |
| 4 | Strong odor |
| 3 | Easily sensible odor |
| 2 | Weak odor of the strength that allows |
| | identification of the source of odor |
| 1 | Weak odor which is almost insensible |
| 0 | No odor |

### Example 4

### Deodorization Rate by Extracts of the Plants of the Genus Rubus of the Family Rosaceae and Green Tea Extract

The comparative tests on the rates of methyl mercaptan deodorization by extracts of the plants of the genus Rubus of the family Rosaceae and a green tea extract in a weakly acidic condition were conducted according to the testing method of Example 2-1. In this Example, the amounts of extracts of the plants of the genus Rubus of the family Rosaceae used were each 1 mg, the amount of the laccase preparation used was also 1 mg (59 U), and the mixture was reacted at 37° C. for 5 minutes.

As a result, as apparent from Table 5 given below, extracts of the plants of the genus Rubus of the family Rosaceae showed a rise of methyl mercaptan deodorization rate by the addition of laccase in the pH range of 4 to 7. Further, the plants of the genus Rubus of the family Rosaceae showed a noticeable increase of the methyl mercaptan deodorization rate by the addition of laccase in the pH range of 4 to 6. It was also confirmed that the plants of the genus Rubus of the family Rosaceae excel green tea used as a control in deodorizing activity in the pH range of 5 to 7.

TABLE 5

Methyl mercaptan
deodorization rate (%)

| | | Addition of | No addition |
|---|---|---|---|
| | | laccase | of laccase |
| pH | Sample name | preparation | preparation |
| pH 4.0 | Tien-cha | 95 | 3 |
| | Raspberry | 95 | 2 |
| | Blackberry | 96 | 1 |
| | Green tea | 90 | 0 |
| | (control) | | |
| pH 5.0 | Tien-cha | 93 | 10 |
| | Raspberry | 95 | 7 |
| | Blackberry | 96 | 8 |
| | Green tea | 61 | 4 |
| | (control) | | |
| pH 6.0 | Tien-cha | 89 | 7 |
| | Raspberry | 90 | 4 |
| | Blackberry | 92 | 4 |
| | Green tea | 50 | 6 |
| | (control) | | |
| pH 7.0 | Tien-cha | 70 | 45 |
| | Raspberry | 52 | 16 |
| | Blackberry | 59 | 25 |
| | Green tea | 40 | 23 |
| | (control) | | |

### Example 5

### Methyl Mercaptan Deodorization Rate and Relative Activity of Enzyme

The relation between methyl mercaptan deodorization rate and relative activity of enzyme was examined according to the testing method of Example 2-1.

In this Example, the amount of the Tien-cha extract contained in the test system was 2.5 mg, the amount of the laccase preparation was 1 mg (59 U), and the reaction was carried out at 37° C. for 5 minutes.

The deodorization rates when laccase was added and that when no laccase was added at pH 4, 4.5, 5, 6 and 7 were plotted. Also plotted was the enzymatic activity of laccase alone at pH 3.5 to 6.

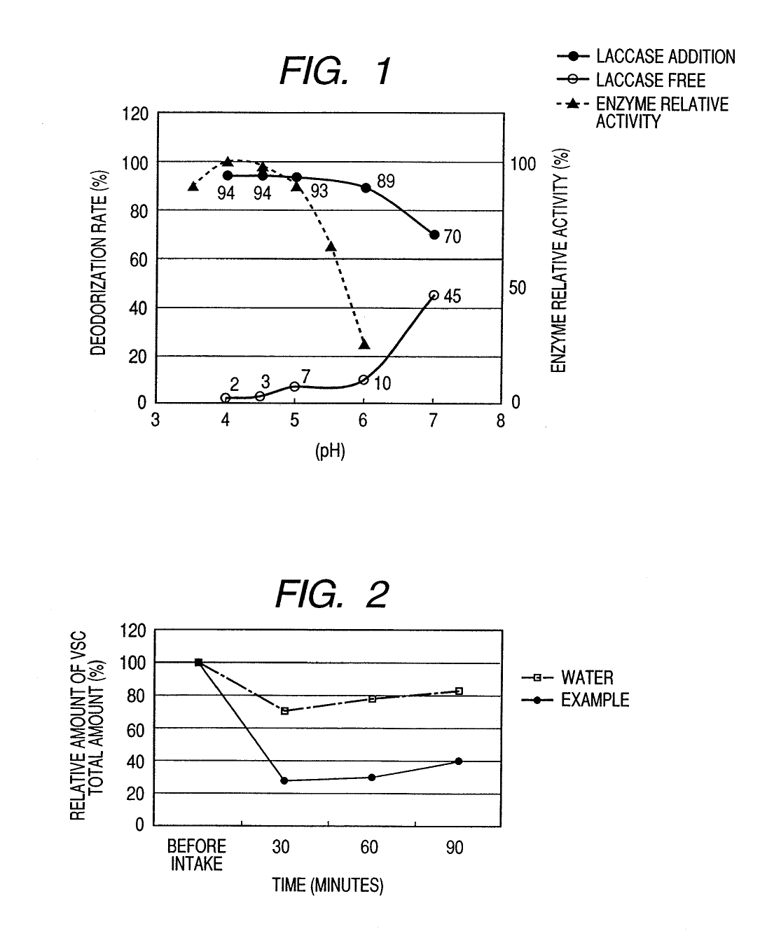

There were obtained the results such as shown in FIG. 1. As apparent from FIG. 1, it was found that the methyl mercaptan deodorization rate is elevated remarkably by the addition of laccase under weak acidity at pH 4 to 7. A noticeable enhancement of methyl mercaptan deodorization rate was also confirmed at pH 6.0 at which the enzyme activity is low. This indicates that the combination of Tien-cha extract and laccase produces a synergistic effect for deodorization of methyl mercaptan.

### Example 6

### Enzyme Concentration and Methyl Mercaptan Deodorization Rate

The relation between enzyme concentration and methyl mercaptan deodorization rate was examined according to the testing method of Example 2-1.

In this Example, the amount of the Tien-cha extract contained in the testing system was 1.0 mg, the amount of the laccase preparation was 0 mg (0 U) to 3.0 mg (177 U), and the reaction was carried out at 37° C. and pH 4.5 for 5 minutes.

As a result, as is apparent from Table 6, there took place a rise of methyl mercaptan deodorization rate with the increase of the content of the laccase preparation added when the content of the Tien-cha extract was fixed at 1.0 mg. Particularly, it was noted that the methyl mercaptan deodorization rate elevated proportionally with the gradual increase of the content of the laccase preparation from 0.1 mg to 1.0 mg. However, no significant additional increase in methyl mercaptan deodorization rate was observed when the amount of the laccase preparation added was increased to 2.0 mg or more.

TABLE 6

| Amount of | Amount | | Amount | Methyl |
|---|---|---|---|---|
| laccase | of | Titer of | of Tien- | mercaptan |
| preparation | laccase | laccase | cha | deodorization |
| added (mg) | (mg) | (U) | extract (mg) | rate (%) |
| 0 | 0 | 0 | 1.0 | 5 |
| 0.1 | 0.03 | 6 | 1.0 | 50 |
| 0.5 | 0.15 | 12 | 1.0 | 86 |
| 1.0 | 0.30 | 59 | 1.0 | 90 |
| 2.0 | 0.60 | 118 | 1.0 | 92 |
| 3.0 | 0.90 | 177 | 1.0 | 93 |

### Example 7

### Deodorizing Effect Test I with Prepared Tablet

Methyl mercaptan deodorization rate was evaluated according to the testing method of Example 2-2 using a tablet prepared according to the testing method of Example 1-5.

As a result, as is seen from Table 7, it was found that the preferred content in the composition of the extract of the plants of the genus Rubus of the family Rosaceae (Tien-cha extract) used in this test is 0.01 to 1.0% by weight, more preferably 0.05 to 0.4% by weight. When the content of the extract of the plants of the genus Rubus of the family Rosaceae in the composition is less than 0.05% by weight, there can not be obtained a desired deodorizing effect. Even if the extract is contained in excess of 0.4% by weight, no proportional rise of deodorizing effect can be obtained. As for the content of laccase used in this test in the composition, it was found that the preferred content of laccase, although variable depending on the properties of the composition, is 0.01 to 1.0% by weight, more preferably 0.015 to 0.12% by weight. It was also found that when the content of laccase in the composition is represented by titer, it should be 5.9×10² U or more and 5.9×10⁴ or less, preferably 3.0×10³ U or more and 2.4×10⁴ U or less, for 100 g of the composition.

When the content of laccase in the composition is less than 0.003% by weight (5.9×10² U/100 g of composition), the desired deodorizing effect can not be obtained. Even if laccase is contained in excess of 0.12% by weight (2.4×10⁴ U/100 g of composition), no corresponding rise of deodorizing effect can be obtained.

TABLE 7

| Tien-cha | Citric | Content of laccase | Titer of laccase | Content of | Methyl mercaptan | |
|---|---|---|---|---|---|---|
| extract | acid | preparation | in 100 g of | laccase in | deodorization | |
| (wt %) | (wt %) | in tablet (wt %) | tablet (U) | tablet (wt %) | rate (%) | pH |
| 0 | 2 | 0 | 0 | 0 | 5 | 5.70 |
| 0.1 | 0 | 0 | 0 | 0 | 28 | 7.48 |
| 0.1 | 2 | 0.1 | 0 | 0 | 6 | 5.68 |
| 0.01 | 2 | 0.1 | 5.9 × 10³ | 0.03 | 15 | 5.75 |
| 0.05 | 2 | 0.1 | 5.9 × 10³ | 0.03 | 25 | 5.57 |
| 0.1 | 2 | 0.01 | 5.9 × 10² | 0.003 | 19 | 5.65 |
| 0.1 | 2 | 0.05 | 3.0 × 10³ | 0.015 | 28 | 5.63 |
| 0.1 | 2 | 0.1 | 5.9 × 10³ | 0.03 | 56 | 5.63 |
| 0.2 | 2 | 0.2 | 1.2 × 10⁴ | 0.06 | 88 | 5.61 |
| 0.3 | 2 | 0.3 | 1.8 × 10⁴ | 0.09 | 92 | 5.64 |
| 0.4 | 2 | 0.4 | 2.4 × 10⁴ | 0.12 | 93 | 5.62 |

### Example 8

### Deodorizing Effect Test II with Prepared Tablet

The breath deodorizing effect was evaluated according to the testing method of Example 2-3 using a tablet prepared according to the testing method of Example 1-6.

The relative amount (%) to the total amount of VSC in the breath with the passage of time was measured and plotted every 30 minutes during a period of 90 minutes after intake of the Example tablet and mouth washing with water.

There was obtained the result shown in FIG. 2. In FIG. 2, pH of the saliva collected after intake of the Example tablet was 5.2. The relative amount to the total amount of VSC in the breath is the relative amount of VSC when the total amount of VSC (hydrogen sulfide, methyl mercaptan and dimethyl monosulfide) in the breath at the start of the test was assumed to be 100%. As is seen from FIG. 2, it was confirmed that the Example tablet is capable of well suppressing the breath odor through the test period of 90 minutes, but in the case of mouth washing with water, the breath odor returns to the level at the start of the test in 90 minutes.

### Example 9

### Garlic Smell Suppressing Effect by Prepared Chewing Gum (Test on Human Being)

Organoleptic evaluation on chewing gum prepared according to the testing method of Example 1-7 was made according to the testing method of Example 3.

As a result, as is seen from Table 8, it was confirmed that the Example chewing gum is capable of most strongly suppressing garlic smell even after intake of the gum. The saliva pH after intake of the Example chewing gum was about 6.0.

TABLE 8

| | | Control | Example |
|---|---|---|---|
| | Gargling | chewing gum | chewing gum |
| Before intake | 1.0 | 1.0 | 1.0 |
| of fried | | | |
| dumpling | | | |
| Immediately | 4.0 | 4.0 | 4.0 |
| after intake | | | |
| of fried | | | |
| dumpling | | | |
| After intake | 3.4 | 2.0 | 1.4 |
| of gum | | | |

### Example 10

A candy, a gummi jelly and a troche were produced according to the following formulations using the extracts prepared according to the methods shown in Examples 1-3 and 1-4.

### Example 10-1

Formulation for candy

| Sugar | 50.0 wt % |
|---|---|
| Syrup | 34.0 |
| Citric acid | 2.0 |
| Raspberry extract | 0.2 |
| Laccase preparation | 0.1 |
| (5.9 × 10⁴ U/g) | |
| Flavor | 0.2 |
| Water | Balance |
| | 100.0 |

### Example 10-2

Formulation for gummi jelly

| Gelatin | 60.0 wt % |
|---|---|
| Syrup | 21.0 |
| Sugar | 8.5 |
| Vegetable fat and oil | 4.5 |
| Mannitol | 3.0 |
| Malic acid | 2.0 |
| Tien-cha extract | 0.2 |
| Laccase preparation | 0.2 |
| (5.9 × 10⁴ U/g) | |
| Flavor | 0.6 |
| | 100.0 |

### Example 10-3

Formulation for troche

| Dextrose | 72.3 wt % |
|---|---|
| Lactose | 15.0 |
| Gum arabic | 6.0 |
| Flavor | 1.0 |
| Sodium monofluorophosphate | 0.7 |
| Blackberry extract | 2.0 |
| Laccase preparation | 1.0 |
| (5.9 × 10⁴ U/g) | |
| Lactic acid | 2.0 |
| | 100.0 |

### INDUSTRIAL APPLICABILITY

The present invention can be desirably applied to a variety of foods and drinks including various types of acid-containing confectionery such as chewing gum, candies, tablets, gummi jellies, etc.

This application claims the benefit of Japanese Patent Application No. 2008-031382, filed Feb. 13, 2008, which is hereby incorporated by reference herein in its entirety.

1. A deodorizing composition comprising a plant of the genus Rubus of the family Rosaceae, laccase and an acid.

2. The deodorizing composition according to claim 1, wherein the plant of the genus Rubus of the family Rosaceae is one or more extracts selected from the group consisting of the extracts of Tien-cha, blackberry and raspberry.

3. The deodorizing composition according to claim 1, wherein the acid is selected from the group consisting of citric acid, lactic acid, malic acid, tartaric acid, fumaric acid and acetic acid.

4. The deodorizing composition according to claim 1, wherein pH in the saliva after intake of the deodorizing composition is in the range of 4.0 to 7.0 inclusive.

5. The deodorizing composition according to claim 1, wherein the content of the plant of the genus Rubus of the family Rosaceae in the deodorizing composition is 0.01% by weight or more and 1.0% by weight or less, and the content of the laccase in the deodorizing composition is 0.01% by weight or more and 1.0% by weight or less.

6. The deodorizing composition according to claim 1, wherein the content of the plant of the genus Rubus of the family Rosaceae in the deodorizing composition is 0.01% by weight or more and 1.0% by weight or less, and the amount of the laccase added, when represented by titer, is 5.9×10² U or above and 5.9×10⁴ U or below, per 100 g of the deodorizing composition.

7. Chewing gum, candies, tablets or gummi jellies comprising a deodorizing composition which includes: a plant of the genus Rubus of the family Rosaceae, laccase and an acid.

8. Chewing gum, candies, tablets or gummi jellies according to claim 7, wherein the plant of the genus Rubus of the family Rosaceae is one or more extracts selected from the group consisting of the extracts of Tien-cha, blackberry and raspberry.

9. Chewing gum, candies, tablets or gummi jellies according to claim 7, wherein the acid is selected from the group consisting of citric acid, lactic acid, malic acid, tartaric acid, fumaric acid and acetic acid.

10. Chewing gum, candies, tablets or gummi jellies according to claim 7, wherein pH in the saliva after intake of the deodorizing composition is in the range of 4.0 to 7.0 inclusive.

11. Chewing gum, candies, tablets or gummi jellies according to claim 7, wherein the content of the plant of the genus Rubus of the family Rosaceae in the deodorizing composition is 0.01% by weight or more and 1.0% by weight or less, and the content of the laccase in the deodorizing composition is 0.01% by weight or more and 1.0% by weight or less.

12. Chewing gum, candies, tablets or gummi jellies comprising a deodorizing composition according to claim 7, wherein the content of the plant of the genus Rubus of the family Rosaceae in the deodorizing composition is 0.01% by weight or more and 1.0% by weight or less, and the amount of the laccase added, when represented by titer, is 5.9×10² U or above and 5.9×10⁴ U or below, per 100 g of the deodorizing composition.
